(19) United States
(12) Patent Application Publication
(10) Pub. No.: US 2024/0091765 A1
(43) Pub. Date: Mar. 21, 2024

(54) MULTISTAGE FLUIDIC DEVICE

(71) Applicants: Todd Michael Haran, Bloomfield, NY (US); Jonas Haran, West Bloomfield, NY (US)

(72) Inventors: Todd Michael Haran, Bloomfield, NY (US); Jonas Haran, West Bloomfield, NY (US)

(21) Appl. No.: 17/964,459

(22) Filed: Oct. 12, 2022

Related U.S. Application Data
(60) Provisional application No. 63/310,517, filed on Feb. 15, 2022.

(51) Int. Cl. B01L 3/00; G01N 33/68

(52) U.S. Cl. CPC B01L 3/502715; G01N 33/6803; B01L 2200/0689; B01L 2300/0832; B01L 2400/0487

(57) ABSTRACT

A multistage fluidic device includes a housing with first and second ends defining an inner volume therebetween. A first sealing member is fixedly secured to the tubular body near the first end. A cannula is disposed through the first sealing member and a plunger is slidably received within the housing. Spaced apart moveable septums are located within the housing and divide the inner volume into a plurality of chambers. Each septum sealingly engages the inner wall of the housing. A first chamber is defined between the first sealing member and a first septum. The first end of the cannula resides within the first chamber. A hollow bore defined by the cannula provides fluid communication between the inner volume of the housing and the environment. A second chamber is defined between the first septum and a second septum and a third chamber is defined between the second septum and the plunger.

### CROSS-REFERENCE TO RELATED APPLICATIONS

This application claims the benefit of U.S. Patent Application No. 63/310,517, filed on Feb. 15, 2022, the contents of which are incorporated by reference in their entirety.

### FIELD OF THE INVENTION

The present invention relates to chemical and biological diagnostics; in particular, to a multistage fluidic device configured to serially deliver diagnostic fluids to a sample; and more particularly to a portable multistage fluidic device that offers in-field screening for selected chemicals or biologics.

### BACKGROUND OF THE INVENTION

Chemical and biological sampling may be a time consuming and labor intensive process requiring multiple steps before completion. Typically, a sequential application of differing fluid reagents is required to capture, wash and then analyze the sample. This process may require skilled lab technicians and expensive lab equipment to perform each function properly. One or both of these may not always be readily available. Thus, to perform analyses at point of care settings or outside of the lab, it is therefore desirable to have a device which assists in collecting, containing, mixing, and dispensing fluids in the proper order while maintaining purity of each of the fluids throughout the process.

Accordingly, there is a need for multistage fluidic device that can serially dispense fluids for chemical and biological sample analyses. There is a further need to provide such a fluidic device in a portable, in-field operable device. The present invention addresses these, as well as other, needs.

### BRIEF SUMMARY OF THE INVENTION

As will be described in more detail below, one aspect of the present invention provides a multistage fluidic device for performing biomarker detection, such as but not limited to, an immunoassay on a sample targeting a selected antigen. The fluidic device comprises a housing having a tubular body with a first end and an opposing second end defining an inner volume therebetween. A first sealing member is fixedly secured to the tubular body at or near the first end of the housing. A cannula passes through the first sealing member such that a first end of the cannula resides in the inner volume of the housing and a second end of the cannula extends outwardly of the housing to the environment. A plunger is slidably received within the housing proximate to the second end and a plurality of spaced apart moveable septums is located within the housing between the first sealing member and the plunger to divide the inner volume into a plurality of chambers. Each septum of the plurality of moveable septums sealingly engages the inner wall of the housing. A first chamber is defined between the first sealing member and a first septum and the first end of the cannula resides within the first chamber. A hollow bore defined by the cannula provides fluid communication between the inner volume of the housing and the environment. A second chamber is defined between the first septum and a second septum and a third chamber is defined between the second septum and the plunger.

In a further aspect of the present invention, the first end of the cannula is tapered to a pointed terminus whereby the pointed terminus is configured to puncture one or more of the moveable septums. The cannula also includes an inlet aperture a spaced distance from the pointed terminus wherein the inlet aperture provides the fluid communication between the inner volume of the housing and the environment. Additionally, each of the moveable septums is comprised of silicone rubber.

In another aspect of the present invention, a first additional septum may be located between the first septum and the second septum so as to define a first additional chamber between the first chamber and the second chamber. A second additional septum may also located between the second septum and the plunger to define a second additional chamber between the second chamber and the third chamber.

In still another aspect of the present invention, the second end of the cannula may be adapted to couple to a filter unit. The filter unit may include a binding agent configured to selectively bind with the antigen in the sample. The first chamber is configured to receive the sample after the sample passes through the filter unit. The second chamber includes a conjugation agent configured to selectively target the bound antigen in the filter unit. The third chamber may then include a detection agent configured to bind to the conjugation agent.

Further, a first additional septum may be located between the first septum and the second septum and define a first additional chamber between the first chamber and the second chamber. The first additional chamber may include a first wash buffer configured to wash the sample before introduction of the conjugation agent in the second chamber to the bound antigen in the filter unit. A second additional septum may be located between the second septum and the plunger and define a second additional chamber between the second chamber and the third chamber. The second additional chamber may include a second wash buffer configured to wash the bound antigen with conjugated agent before introduction of the detection agent in the third chamber to the bound antigen with conjugated agent in the filter unit.

The present invention may further provide a method for performing biomarker detection, such as via an immunoassay, on a sample targeting a selected antigen. The method may include the steps of a) providing the above-described multistage fluidic device; b) placing the second end of the cannula into a sample container holding the sample; c) withdrawing the plunger to draw a volume of sample into the first chamber of the fluidic device; d) depressing the plunger a first distance to cause the first septum to rupture against the first end of the cannula whereby a second chamber fluid within the second chamber is introduced into the first chamber; e) further depressing the plunger a second distance to cause the second septum to rupture against the first end of the cannula whereby a third chamber fluid within in third chamber is introduced into the first chamber; and f) interrogating the sample to determine whether the selected antigen is present in the sample.

In another aspect of the present invention, the method may further comprise the step of placing a filter unit onto the second end of the cannula before step b), wherein the plunger draws the volume of sample through the filter unit and into the first chamber of the fluidic device in step c). The fluidic device may further comprise a first additional septum located between the first septum and the second septum to thereby define a first additional chamber between the first chamber and the second chamber. The first additional chamber includes a first additional fluid such that the method further comprises depressing the plunger until the first additional septum is ruptured whereby the first additional fluid comingles with the sample within the filter unit before introduction of the second chamber fluid in step d). Still further, the fluidic device may also comprise a second additional septum located between the second septum and the plunger to thereby define a second additional chamber between the second chamber and the third chamber. The second additional chamber includes a second additional fluid and the method further comprises depressing the plunger until the second additional septum is ruptured whereby the second additional fluid comingles with the sample within the filter unit before introduction of the third chamber fluid in step e).

The present invention may further provide an alternative multistage fluidic device for performing biomarker detection, such as an immunoassay, on a liquid sample targeting a selected antigen. The alternative fluidic device may include an outer housing comprising a tubular body having a closed first end and an open opposing second end. The tubular body defines an inner volume. A cannula passes through the closed first end of the outer housing such that a first end of the cannula resides in the inner volume of the outer housing and a second end of the cannula extends outwardly of the outer housing to the environment. The device also includes a plurality of nesting tubular members, each having a respective closed first end and open opposing second end. The outermost tubular member is sealably and slidably received within the open opposing second end of the first housing. Each successive tubular member is dimensioned to be sealably and slidably received within the respective open opposing second end of its immediately preceding tubular member. A plunger is then sealably and slidably received within an innermost tubular member of the plurality of tubular members. The closed first end of the outer housing is spaced apart from the closed first end of the outermost tubular member so as to define a first chamber therebetween. The first end of the cannula resides within the first chamber and a hollow bore defined by the cannula provides fluid communication between the inner volume of the outer housing and the environment. The closed first end of each successive nesting tubular member is spaced apart from the closed first end of its preceding nesting tubular member to define a series of respective chambers therebetween. The first chamber and each respective chamber of the series of respective chambers may then be filled with a non-compressible fluid.

In another aspect of the present invention, the method may further include having the first end of the cannula being tapered to a pointed terminus whereby the pointed terminus is configured to puncture the closed first ends of the plurality of nesting tubular members. Also the cannula includes an inlet aperture a spaced distance from the pointed terminus such that the inlet aperture provides the fluid communication between the inner volume of the housing and the environment. Alternatively, each of the closed first ends of the plurality of nesting tubular members may include an actuatable valve; wherein a respective actuatable valve on the outermost tubular member is actuated by the first end of the cannula and each successive actuatable valve of each successive nesting tubular member is actuated by the actuatable valve of its immediately preceding nesting tubular member.

Additional objects, advantages and novel features of the present invention will be set forth in part in the description which follows, and will in part become apparent to those in the practice of the invention, when considered with the attached figures.

### BRIEF DESCRIPTION OF THE DRAWINGS

The accompanying drawings form a part of this specification and are to be read in conjunction therewith, wherein like reference numerals are employed to indicate like parts in the various views, and wherein:

FIG. 1 is an exploded schematic representation of an embodiment of an exemplary multistage fluidic device in accordance with an aspect of the present invention;

FIG. 2 is a schematic of the embodiment of the exemplary multistage fluidic device shown in FIG. 1;

FIGS. 3-9 show a step-wise exemplary process and method of using the exemplary embodiment of a multistage fluidic device shown in FIGS. 1 and 2;

FIG. 10 is a schematic representation of an alternative embodiment of an exemplary multistage fluidic device in accordance with another aspect of the present invention;

FIG. 11 is a schematic representation of the exemplary multistage fluidic device shown in FIG. 10 after actuation and dispensing of a first fluid chamber;

FIG. 12 is a schematic representation of yet another embodiment of an exemplary multistage fluidic device in accordance with another aspect of the present invention;

FIG. 13 is a schematic representation of the embodiment of an exemplary multistage fluidic device shown in FIG. 12 after actuation and dispensing of a first fluid chamber;

FIG. 14 is a schematic representation of a further embodiment of an exemplary multistage fluidic device in accordance with another aspect of the present invention; and

FIG. 15 is a schematic representation of still another embodiment of an exemplary multistage fluidic device in accordance with another aspect of the present invention.

### DETAILED DESCRIPTION OF THE INVENTION

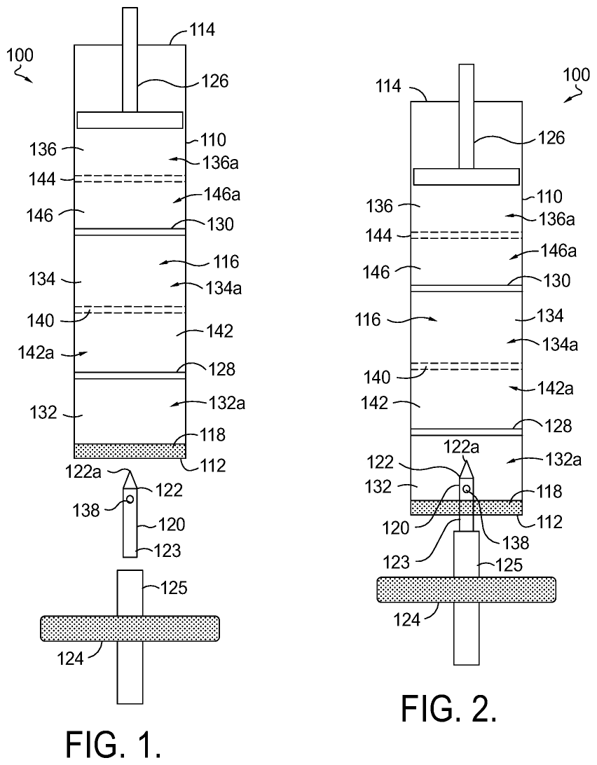

Referring to the drawings in detail, and specifically to FIGS. 1 and 2, an exemplary embodiment of a multistage fluidic device 100 is shown. Fluidic device 100 includes a housing 110 that is generally tubular shaped, such as but not limited to, a syringe. Housing 110 has a first end 112 and a second end 114 defining an inner volume 116 therebetween. A first sealing member 118 is fixedly secured to housing 110 at or near first end 112. In one aspect, first sealing member 118 is of a unitary construction with housing 110. In another aspect, first sealing member 118 is a separate structure that is secure to housing 110, such as internally through, for instance, a friction fit, or externally, such as via a luer-type locking mechanism. At least a portion of first sealing member 118 is frangible so as to permit passage of a cannula 120 therethrough. It should also be noted that cannula 120 may be integrally formed with first sealing member 118, and may further be integrally formed with housing 110. In any event, cannula 120 includes a first end 122 which resides within inner volume 116 and a second end 123 which extends outwardly of housing 110. Second end 123 of cannula 120 may be adapted to mount with a top end 125 of a filter unit 124, such as via a friction fit, luer-lock or other securement.

With continued reference to FIGS. 1 and 2, the opposing second end 114 of housing 110 is configured to slidably receive plunger 126 therein. A plurality of spaced apart septums, such as, for example a first septum 128 and a second septum 130 are located within inner volume 116 of housing 110 and divide inner volume 116 into a corresponding number of chambers. In the instance where fluidic device 100 includes first and second septums 128, 130, a first chamber 132 is defined between first sealing member 118 and first septum 128, a second chamber 134 is defined between first septum 128 and second septum 130 and a third chamber 136 is defined between second septum 130 and plunger 126.

Each septum 128, 130 seals against an inner surface of housing 110 while each of chambers 132, 134, 136 may be filled with a respective compressible and/or non-compressible fluid 132a, 134a, 136a. It should be understood that fluids 132a, 134a, 136a may be the same fluid, different fluids relative to one another, or any combination thereof. As a result, each septum 128, 130 travels within housing 110 upon withdrawal and depressing of plunger 126 so as to maintain the nominal fluid volume or air mass of each chamber 132, 134, 136. In a preferred embodiment, at least a portion of each septum 128, 130 is constructed of a frangible material, such as, but not limited to, silicone rubber. Each of the moveable septums may also be comprised of a frangible silicone rubber portion supported by an injection molded frame which may provide structural support to the septum.

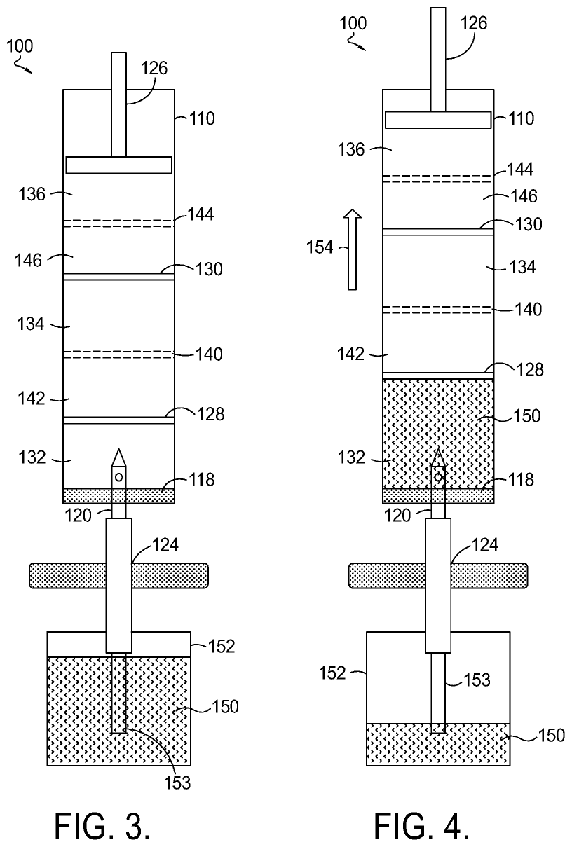

As will be described in greater detail below with regard to FIGS. 3-9, first end 122 of cannula 120 may be constructed so as to have a pointed terminus 122a which is adapted to pierce/puncture septum 128, 130 as plunger 126 is depressed. Cannula 120 may also include an inlet aperture 138 located at, or proximate to but a spaced distance from, pointed terminus 122a. Inlet aperture 138 provides fluid communication from inner volume 116 to the environment (or to filter unit 124 if mounted onto cannula 120) through the hollow body of cannula 120. As a result, fluid within chambers 132, 134, 136 may be serially dispensed from housing 110 upon depressing of plunger 126 and subsequent sequential communication of chambers 134, 136 with inlet aperture 138 following puncture of their respective septums 128, 130.

As further shown in FIGS. 1 and 2, any number of septums (n) may be included within housing 110 to create a corresponding number of chambers (n+1). By way of example and without limitation thereto, a first additional septum 140 may be located between first septum 128 and second septum 130 so as to define a first additional chamber 142. First additional chamber 142 may be filled with any desired fluid 142a as desired or necessary to conduct the chemical or biological analysis. Similarly, a second additional septum 144 may be located between second septum 130 and plunger 126 so as to define a second additional chamber 146, which may be filled with a fluid 146a, as will be discussed in greater detail below.

Turning now to FIGS. 3-9, an exemplary step-wise method for performing biomarker detection, such as but not limited to an immunoassay, on a sample targeting a selected antigen is schematically shown. As shown in FIG. 3, multistage fluidic device 100, including cannula 120 and filter unit 124 mounted thereto, is inserted into a fluid sample 150 in a sample container 152, such as via sample draw tube 153. It should be noted that, while shown and described as being preassembled to include filter unit 124, fluidic device 100 may be inserted directly into fluid sample 150 solely via cannula 120.

With reference to FIG. 4, plunger 126 is withdrawn within housing 110 in a direction 154 so as to draw fluid sample 150 through filter unit 124 into first chamber 132. Again, note that some applications may exclude filter unit 124 so that fluid sample 150 may be drawn directly into first chamber 132 via cannula 120. By way of example and without limitation thereto, first chamber fluid 132a may be include a chemical reagent configured to operate on fluid sample 150 or may simply be a dilution buffer. Typically, this mixed solution in first chamber 132 would then be dispensed through filter unit 124 after filter unit 124 is coupled to cannula 120 where a physical-particulate filter medium within filter unit 124 would collect the sample analyte. Some applications, like soil sampling, might require the sequence described above, but with an additional small pore pre-filter unit for particulate removal before passing first chamber fluid 132a/sample 150 through filter unit 124.

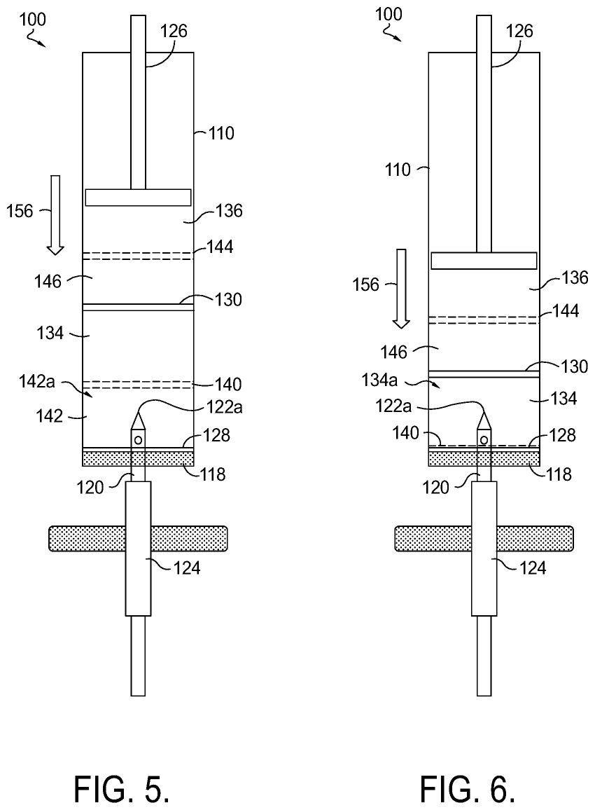

As shown in FIG. 5, following any desired length of time or inversion/fluid mixing protocols, plunger 126 may then be depressed in a direction 156 until first chamber 132 is fully dispensed from fluidic device 100 through filter unit 124 and first septum 128 has been punctured by pointed terminus 122a of cannula 120. If needed, filter unit 124 may be coupled to cannula 120 prior to depression of plunger 126. The dispensed fluid sample/first chamber fluid may be collected for analysis or appropriately discarded as waste. At this point, if present in the sample, the target antigen will be bound to affinity media within filter unit 124. By way of example and without limitation, the affinity media may be a bead or surface treated to include a capture antibody selective to the antigen of interest, or may be a particulate filter, chromatographic column, etc., depending upon the use application.

As shown in FIG. 6, plunger 126 has been further depressed in direction 156 until first additional septum 140 has been punctured by pointed terminus 122a such that first additional fluid 142a (FIG. 5) within first additional chamber 142 has been dispensed through filter unit 124. Again, first additional fluid 142a may be collected for analysis or discarded as waste. In one exemplary embodiment, first additional fluid 142a may comprise a first wash buffer configured to wash the filter unit 124 and any antigen bound to the filter media so as to reduce or minimize chemical interference during subsequent serial dispensing of fluid from fluidic device 100.

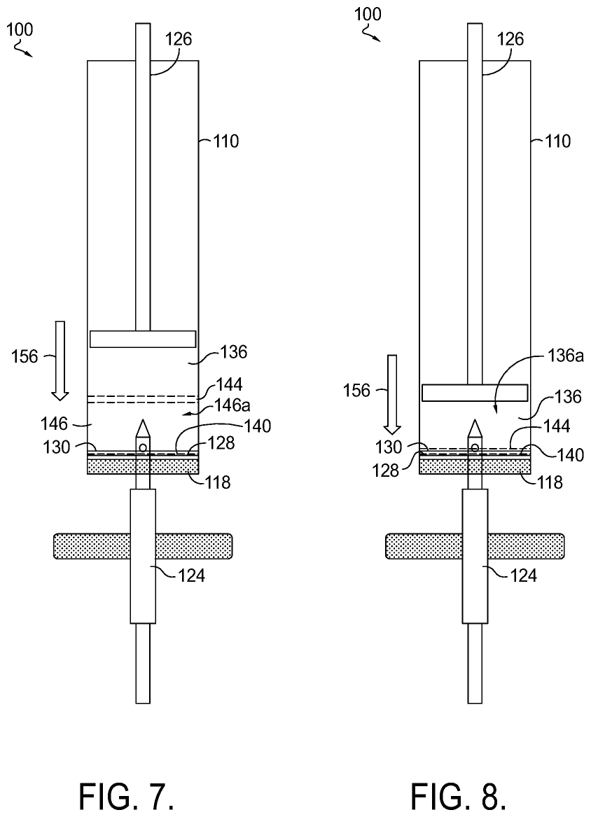

As shown in FIG. 7, plunger 126 has been further depressed in direction 156 until second septum 130 has been punctured such that second chamber fluid 134a (FIG. 6) within second chamber 134 has been dispensed through filter unit 124. Again, second chamber fluid 134a may be collected for analysis or discarded as waste. In one exemplary embodiment, second chamber fluid 134a may include a secondary detection antibody configured to bind with the bound antigen in filter unit 124 in an antibody sandwich.

As shown in FIG. 8, plunger 126 has been further depressed in direction 156 until second additional septum 144 has been punctured such that second additional fluid 146a (FIG. 7) within second additional chamber 146 has been dispensed through filter unit 124. Again, second additional fluid 146a may be collected for analysis or discarded as waste. In one exemplary embodiment, second additional fluid 146a may comprise a second wash buffer configured to wash the filter unit 124 and any antigen sandwich bound to the filter media so as to reduce or minimize chemical interference during subsequent serial dispensing of fluid from fluidic device 100.

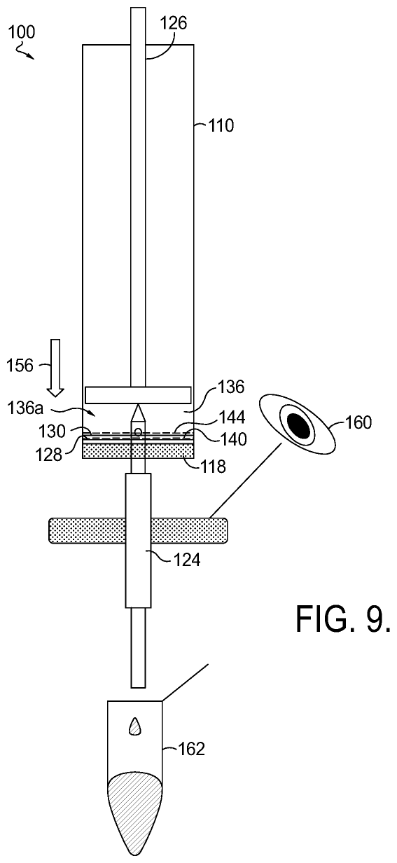

As shown in FIG. 9, plunger 126 has been fully depressed in direction 156 such that third chamber fluid 136a (FIGS. 8 and 9) within second chamber 136 has been dispensed through filter unit 124. In one exemplary embodiment, third chamber fluid 136a may include a colorimetric substrate configured to generate a color signal when bound with the antibody sandwich in filter unit 124. This color signal may be detectable by the naked eye 160, or may require alternative analysis. Third chamber fluid 136a may additionally or alternatively include an elution buffer whereby the eluted antibody sandwich may be collected, such as in an Eppendorf tube or other collection vial or plate 162, for visual and/or machine analysis.

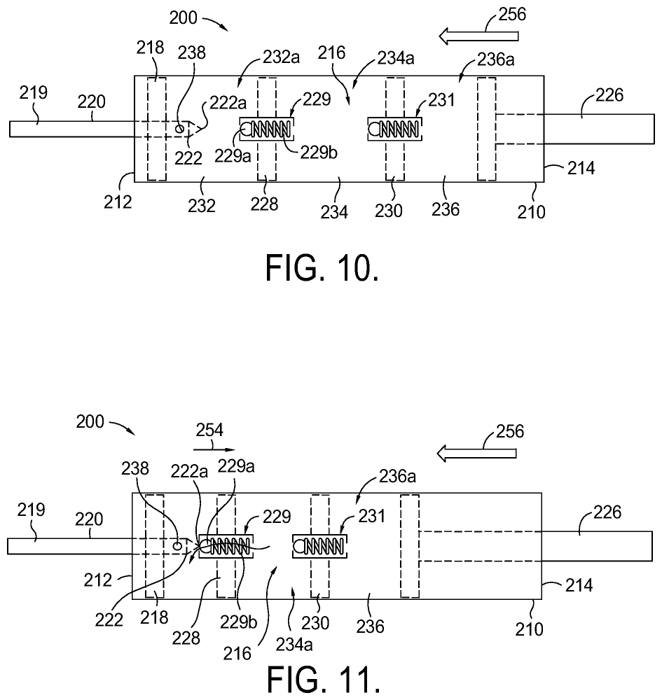

Turning now to FIGS. 10 and 11, an exemplary embodiment of an alternative multistage fluidic device 200 is shown. Fluidic device 200 is substantially the same as fluidic device 100 described above, except for the actuation of the various septums located within the device. Fluidic device 200 generally comprises a housing 210 that is generally tubular shaped, such as but not limited to, a syringe. Housing 210 has a first end 212 and second end 214 defining an inner volume 216 therebetween. A first sealing member 218 is fixedly secured to housing 210 at or near first end 212. At least a portion of first sealing member 218 is frangible so as to permit passage of cannula 220 therethrough. Cannula 220 includes a first end 222 which resides within inner volume 216 and a second end 223 which extends outwardly of housing 210. Although not shown, second end 223 of cannula 220 may be adapted to mount a filter unit, such as filter unit 124, thereto similar to second end 123 of cannula 120.

The opposing second end 214 of housing 210 is configured to slidably receive plunger 226 therein. A plurality of spaced apart septums, such as, for example a first septum 228 and second septum 230, are located within inner volume 216 of housing 210 and divide inner volume 216 into a corresponding number of chambers. In the instance that first and second septums 228, 230 are included, a first chamber 232 is defined between first sealing member 218 and first septum 228, a second chamber 234 is defined between first septum 228 and second septum 230 and a third chamber 236 is defined between second septum 230 and plunger 226. Each septum 228, 230 seals against inner surface of housing 210 while each of chambers 232, 234, 236 may be filled with a respective non-compressible fluid 232a, 234a, 236a.

With further reference to FIG. 11, first end 222 of cannula 220 may be constructed so as to have a pointed terminus 222a. Cannula 220 may also include an inlet aperture 238 located at, or proximate to but a spaced distance from, pointed terminus 222a. Inlet aperture 238 provides fluid communication from inner volume 216 to the environment through the hollow body of cannula 220. However, unlike fluidic device 100, fluidic device 200 includes septums 228, 230 which include a respective selectively actuatable check valve 229, 231 thereon. Thus, as plunger 226 is depressed in a direction 256, pointed terminus 222a may engage a ball 229a of check valve 229 to drive ball 229a against a valve spring 229b in a direction 254 so as to open check valve 229. As a result, fluid 234a in second chamber 234 may be dispensed through cannula 220. Fluid 236a may be serially dispensed from chamber 236 upon continued depressing of plunger 226 in direction 256, whereby check valve 229 may engage check valve 231 so as to open check valve 231 to dispense fluid 236a.

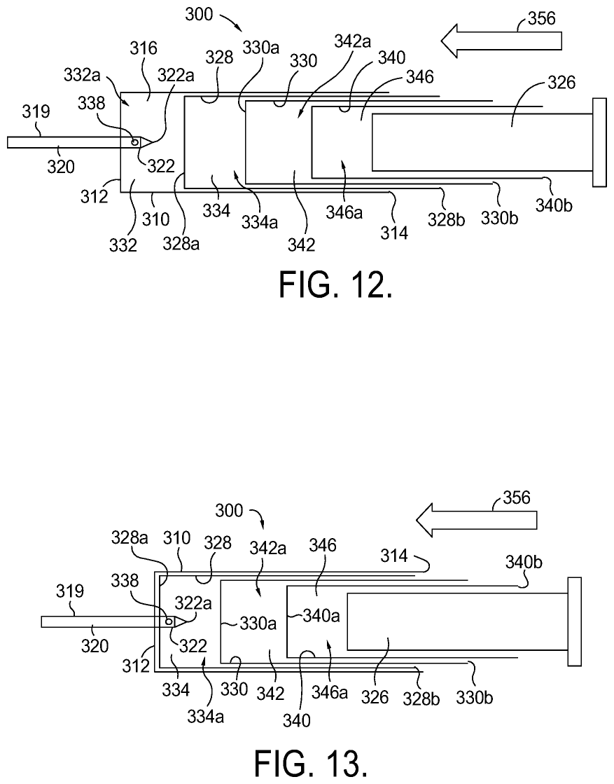

Turning now to FIGS. 12 and 13, an exemplary embodiment of still another alternative multistage fluidic device 300 is shown. Fluidic device 300 generally comprises an outer housing 310 that is generally tubular shaped with a closed first end 312 and an open opposing second end 314 defining an inner volume 316 therebetween. Cannula 320 passes through closed first end 312 and includes a first end 322 which resides within inner volume 316 and a second end 319 which extends outwardly of outer housing 310. Although not shown, second end 219 of cannula 220 may be adapted to mount a filter unit, such as filter unit 124, thereto similar to second end 123 of cannula 120. First end 322 of cannula 320 may be constructed so as to have a pointed terminus 322a as will be discussed in greater detail below.

Open opposing second end 314 is configured to sealably and slidably receive a first nesting tubular member 328 therein. First nesting tubular member 328 has a closed first end 328a and an open opposing second end 328b. Closed first end 328a is located a spaced distance from closed first end 312 of the outer housing 310 so as to define a first chamber 332 which may be filled with a first chamber fluid 332a. Open opposing second end 328b of first nesting tubular member 328 is configured to sealably and slidably receive a second nesting tubular member 330 therein. Second nesting tubular member 330 has a closed first end 330a and an open opposing second end 330b. Closed first end 330a is located a spaced distance from closed first end 328a of first nesting tubular member 328 so as to define a second chamber 334 which may be filled with a second chamber fluid 334a.

One or more additional nesting tubular members (e.g., a third nesting tubular member 340) may be included within fluidic device 300 as desired, so as create any desired number of fluid chambers (e.g., a third chamber 342 filled with a third chamber fluid 342a). The open opposing second end of the innermost nesting tubular member (i.e., a second end 340b of third nesting tubular member 340 as shown in FIG. 11) may then sealably and slidably receive a plunger 326 therein. Plunger 326 may be located a spaced distance from a closed first end 340a of third nesting tubular member 340 so as to define a fourth chamber 346 which may be filled with a fourth chamber fluid 346a. It should be understood that each of the first, second, third and/or fourth chamber fluids 332a, 334a, 342a, 346a may be the same as one or more of the other chamber fluids, or different than one or more of the other chamber fluids. In other words, any combination of chamber fluids is contemplated in the present invention.

As shown in FIG. 13, in a first exemplary embodiment of fluidic device 300, each of closed first ends 328a, 330a and 340a may be constructed to include a frangible septum similar to septums 128, 130, 140 described above with regard to fluidic device 100. Frangible first ends 328a, 330a and 340a may then be punctured by pointed terminus 322a of cannula 320 as plunger 326 is depressed in direction 356. As a result, fluid 332a, 334a, 342a, 346a within respective chambers 332, 334, 342, 346 may be serially dispensed from fluidic device 300 upon depressing of plunger 326 as each tubular member 328, 330, 340 is telescopically received within outer housing 310.

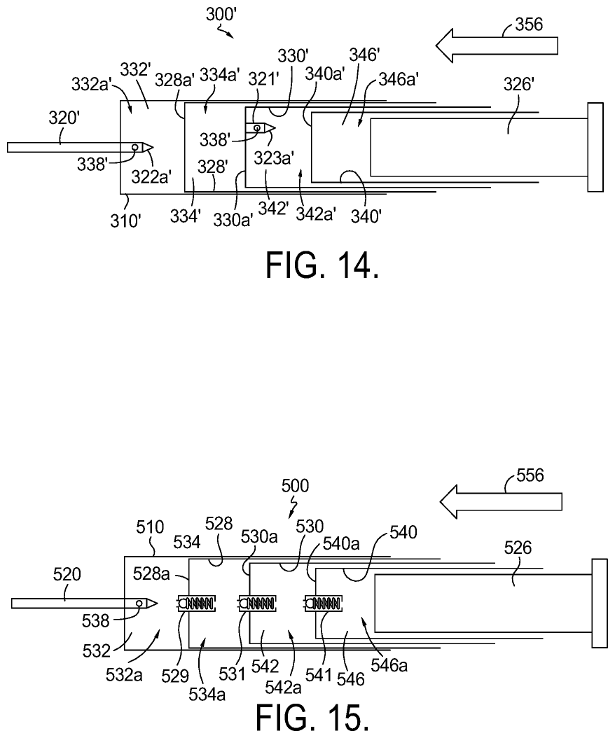

As shown in FIG. 14, in an alternative exemplary embodiment of fluidic device 300′, each of closed first ends 328a′, 330a′ and 340a′ may be constructed to include a frangible septum similar to first ends 328a, 330a, 340a. Frangible first ends 328a′ and 330a′ may be punctured by pointed terminus 322a′ of cannula 320′ as plunger 326′ is depressed in direction 356. However, closed end 330a′ may also include a supplemental hollow cannula 321′ with pointed terminus 323a′ configured to puncture closed end 340a′. As a result, fluid 332a′, 334a′ 342a′ 346a′ within respective chambers 332′, 334′, 342′, 346′ may be serially dispensed from fluidic device 300′ through cannula 320′ via aperture 338′ upon depressing of plunger 326′ as each tubular member 328′, 330′, 340′ is telescopically received within outer housing 310′.

In a further exemplary embodiment shown in FIG. 15, fluidic device 500 may be substantially identical to fluidic device 300 with the exception that fluidic device 500 may comprise an outer housing 510 receiving nesting tubular members 528, 530, 540 each having a respective actuatable check valve 529, 531, 541 within its respective closed first end 528a, 530a, 540a similar to check valves 229, 231 described above with regard to fluidic device 200. Check valves 529, 531, 541 may be similarly actuated upon depressing of plunger 526 in direction 556 to dispense fluids 532a, 534a, 542a, 546a within respective chambers 532, 534, 542, 546 through cannula 520 via aperture 538 as each tubular member 528, 530, 540 is telescopically received within outer housing 510.

From the foregoing, it will be seen that this invention is one well adapted to attain all the ends and objects hereinabove set forth together with other advantages which are obvious and which are inherent to the device described herein. It will be understood that certain features and sub combinations are of utility and may be employed without reference to other features and sub combinations. This is contemplated by and is within the scope of the claims. Since many possible embodiments of the invention may be made without departing from the scope thereof, it is also to be understood that all matters herein set forth or shown in the accompanying drawings are to be interpreted as illustrative and not limiting.

The constructions described above and illustrated in the drawings are presented by way of example only and are not intended to limit the concepts and principles of the present invention. As used herein, the terms “having” and/or “including” and other terms of inclusion are terms indicative of inclusion rather than requirement. Further, it should be understood that the use of the terms “module” and “component” herein are interchangeable and shall have the same meaning.

While the invention has been described with reference to preferred embodiments, it will be understood by those skilled in the art that various changes may be made and equivalents may be substituted for elements thereof to adapt to particular situations without departing from the scope of the invention. Therefore, it is intended that the invention not be limited to the particular embodiments disclosed as the best mode contemplated for carrying out this invention, but that the invention will include all embodiments falling within the scope and spirit of the appended claims.

What is claimed is:

1. A multistage fluidic device for performing biomarker detection of a sample targeting a selected antigen, the fluidic device comprising:
a) a housing comprising a tubular body including a first end and a second end, wherein the tubular body includes an inner surface that defines an inner volume;
b) a first sealing member fixedly secured to the tubular body at or near the first end;
c) a cannula including a first end and a second end, wherein the cannula is disposed through the first sealing member, wherein the first end of the cannula resides in the inner volume of the housing, and wherein the second end of the cannula extends outwardly of the housing to an external environment;
d) a plunger slidably received within the inner volume of the housing proximate to the second end; and
e) a plurality of spaced apart moveable septums located within the inner volume of the housing between the first sealing member and the plunger to divide the inner volume into a plurality of chambers, wherein each of the plurality of moveable septums are sealingly engaged with the inner surface of the housing,
wherein the plurality of spaced apart moveable septums includes a first septum and a second septum,
wherein the plurality of chambers includes a first chamber, a second chamber, and a third chamber,
wherein the first chamber is defined between the first sealing member and the first septum, wherein the second chamber is defined between the first septum and the second septum, and wherein the third chamber is defined between the second septum and the plunger, and
wherein the first end of the cannula resides within the first chamber and a hollow bore defined by the cannula provides fluid communication between the inner volume of the housing and the external environment.

2. The device in accordance with claim 1 wherein the first end of the cannula is tapered to a pointed terminus, and wherein the pointed terminus is configured to puncture at least one of the first septum and the second septum.

3. The device in accordance with claim 2 wherein the cannula defines an inlet aperture located at a spaced distance from the pointed terminus, and wherein the inlet aperture provides the fluid communication between the inner volume of the housing and the external environment.

4. The device in accordance with claim 1 wherein each of the plurality of spaced apart moveable septums is comprised of silicone rubber.

5. The device in accordance with claim 4 wherein each of the plurality of spaced apart moveable septums is comprised of silicone rubber supported by an injection molded frame providing structural support to the respective septum.

6. The device in accordance with claim 1 further comprising a third septum located within the inner volume and disposed between the first septum and the second septum, wherein the third septum divides the second chamber into a fourth chamber and a fifth chamber.

7. The device in accordance with claim 6 further comprising a fourth septum located within the inner volume and disposed between the second septum and the plunger, wherein the fourth septum divides the third chamber into a sixth chamber and a seventh chamber.

8. The device in accordance with claim 1 wherein the second end of the cannula is adapted to couple to a filter unit, wherein the filter unit includes a binding agent configured to selectively bind with the selected antigen, wherein the first chamber is configured to receive the sample after the sample passes through the filter unit, wherein the second chamber includes a conjugation agent configured to selectively target the bound antigen, wherein the third chamber includes a detection agent configured to bind to the conjugation agent.

9. The device in accordance with claim 8 further comprising a third septum located within the inner volume and disposed between the first septum and the second septum, wherein the third septum divides the second chamber into a fourth chamber and a fifth chamber, wherein the fourth chamber includes a first wash buffer configured to wash the sample before introduction of the conjugation agent in the fifth chamber to the bound antigen in the filter unit.

10. The device in accordance with claim 9 further comprising a fourth septum located within the inner volume and disposed between the second septum and the plunger, wherein the fourth septum divides the third chamber into a sixth chamber and a seventh chamber, wherein the sixth chamber includes a second wash buffer configured to wash the bound antigen with conjugated agent before introduction of the detection agent in the seventh chamber to the bound antigen with conjugated agent in the filter unit.

11. The device in accordance with claim 1 wherein each of the plurality of spaced apart moveable septums includes an actuatable valve, wherein a respective actuatable valve on the first septum is actuated by the first end of the cannula, and wherein each successive actuatable valve of each successive spaced apart moveable septum is actuated by the actuatable valve of its immediately preceding spaced apart moveable septum.

12. A method for performing biomarker detection of a sample targeting a selected antigen, the method comprising:
a) providing a multistage fluidic device including:
i) a housing comprising a tubular body including a first end and a second end, wherein the tubular body includes an inner surface that defines an inner volume;
ii) a first sealing member fixedly secured to the tubular body at or near the first end;
iii) a cannula including a first end and a second end, wherein
the cannula is disposed through the first sealing member, wherein the first end of the cannula resides in the inner volume of the housing, and wherein the second end of the cannula extends outwardly of the housing to an external environment;
iv) a plunger slidably received within the inner volume of the housing proximate to the second end; and
v) a plurality of spaced apart moveable septums located within the inner volume of the housing between the first sealing member and the plunger to divide the inner volume into a plurality of chambers, wherein each of the plurality of moveable septums are sealingly engaged with the inner surface of the housing,
wherein the plurality of spaced apart moveable septums includes a first septum and a second septum,
wherein the plurality of chambers includes a first chamber, a second chamber, and a third chamber,
wherein the first chamber is defined between the first sealing member and the first septum, wherein the second chamber is defined between the first septum and the second septum, and wherein the third chamber is defined between the second septum and the plunger, and
wherein the first end of the cannula resides within the first chamber and a hollow bore defined by the cannula provides fluid communication between the inner volume of the housing and the external environment;
b) providing a filter unit in fluid communication with the hollow bore of the cannula;
c) placing the second end of the cannula into a sample container holding the sample;
d) withdrawing the plunger to draw a volume of the sample from the second end of the cannula to the first end of the cannula and into the first chamber of the fluidic device;
e) depressing the plunger a first distance to dispense the volume of the sample through the filter unit;
f) further depressing the plunger a second distance to cause the first septum to rupture against the first end of the cannula, wherein a second chamber fluid disposed within the second chamber is dispensed through the filter unit;
g) further depressing the plunger a third distance to cause the second septum to rupture against the first end of the cannula, wherein a third chamber fluid disposed within in third chamber is dispensed through the filter unit; and
h) interrogating the sample to determine whether the selected antigen is present in the sample.

13. The method in accordance with claim 12 wherein the first end of the cannula is tapered to a pointed terminus, and wherein the pointed terminus is configured to puncture at least one of the first septum and the second septum.

14. The method in accordance with claim 13 wherein the cannula defines an inlet aperture located at a spaced distance from the pointed terminus, and wherein the inlet aperture provides the fluid communication between the inner volume of the housing and the external environment.

15. The method in accordance with claim 12 wherein the fluidic device further comprises a third septum located within the inner volume and disposed between the first septum and the second septum, wherein the third septum divides the second chamber into a fourth chamber and a fifth chamber, wherein the fourth chamber includes the second chamber fluid, wherein the fifth chamber includes a fifth chamber fluid, and wherein the method further comprises depressing the plunger until the third septum is ruptured wherein the fifth chamber fluid comingles with the sample within the filter unit after step f) and before step g).

16. The method in accordance with claim 15 wherein the fluidic device further comprises a fourth septum located within the inner volume and disposed between the second septum and the plunger, wherein the fourth septum divides the third chamber into a sixth chamber and a seventh chamber, wherein the sixth chamber includes the third chamber fluid, wherein the seventh chamber includes a seventh chamber fluid, and wherein the method further comprises depressing the plunger until the fourth septum is ruptured wherein the seventh chamber fluid comingles with the sample within the filter unit after step g).

17. The method in accordance with claim 12 further comprising the step of placing the filter unit onto the second end of the cannula.

18. The method in accordance with claim 17 wherein the first end of the cannula is tapered to a pointed terminus, and wherein the pointed terminus is configured to puncture each of the moveable septums.

19. The method in accordance with claim 18 wherein the cannula includes an inlet aperture a spaced distance from the pointed terminus, wherein the inlet aperture is in fluid communication with the hollow bore.

20. The method in accordance with claim 12 further comprising the step of providing a first chamber fluid in the first chamber before step d).

21. A multistage fluidic device for performing biomarker detection of a liquid sample targeting a selected antigen, the fluidic device comprising:
a) an outer housing comprising a tubular body including a closed first end and an open second end, wherein the tubular body includes an inner surface that defines an inner volume;
b) a cannula including a first end and a second end, wherein the cannula is disposed through the closed first end of the outer housing, wherein the first end of the cannula resides in the inner volume of the outer housing, and wherein the second end of the cannula extends outwardly of the outer housing to an external environment;
c) a plurality of nesting tubular members each having a respective closed first end and an open second end, wherein an outermost tubular member of the plurality of nesting tubular members is sealably and slidably received within the open second end of the outer housing, and wherein each successive tubular member plurality of nesting tubular members is dimensioned to be sealably and slidably received within the respective open second end of its immediately preceding tubular member;
d) a plunger sealably and slidably received within an innermost tubular member of the plurality of nesting tubular members; and
wherein the closed first end of the outer housing is spaced apart from the closed first end of the outermost tubular member so as to define a first chamber therebetween, wherein the first end of the cannula resides within the first chamber and a hollow bore defined by the cannula provides fluid communication between the inner volume of the outer housing and the external environment,
wherein the closed first end of each successive nesting tubular member is spaced apart from the closed first end of its preceding nesting tubular member to define a series of respective chambers therebetween, and
wherein the first chamber and each respective chamber of the series of respective chambers is filled with a fluid.

22. The multistage fluidic device in accordance with claim 21 wherein the first end of the cannula is tapered to a pointed terminus, and wherein the pointed terminus is configured to puncture the closed first ends of the plurality of nesting tubular members.

23. The multistage fluidic device in accordance with claim 22 wherein the cannula defines an inlet aperture located at a spaced distance from the pointed terminus, and wherein the inlet aperture provides the fluid communication between the inner volume of the housing and the external environment.

24. The multistage fluidic device in accordance with claim 21 wherein each of the closed first ends of the plurality of nesting tubular members includes an actuatable valve, wherein a respective actuatable valve on the outermost tubular member is actuated by the first end of the cannula, and wherein each successive actuatable valve of each successive nesting tubular member is actuated by the actuatable valve of its immediately preceding nesting tubular member.
